(19) United States
(12) Patent Application Publication
(10) Pub. No.: US 2009/0163925 A1
(43) Pub. Date: Jun. 25, 2009

(54) INSTRUMENT FOR MEASURING THE STABILITY OF THE CERVICAL SPINE

(76) Inventor: Arnold Keller, Kayhude (DE)

Correspondence Address: MORRISON & FOERSTER LLP, 1650 TYSONS BOULEVARD, SUITE 400, MCLEAN, VA, 22102, US

(21) Appl. No.: 12/281,950

(22) PCT Filed: Mar. 6, 2007
(86) PCT No.: PCT/EP07/01902

§ 371 Date: Sep. 5, 2008

(51) Int. Cl. A61B 17/58

(52) U.S. Cl. 606/102

(57) ABSTRACT

An instrument for measuring the stability of the cervical spine includes two arms each having, at one end, a contact plate configured for insertion into a space between two adjacent vertebral bodies, and, at an end opposite to the one end, an actuating member. The arms are movable relative to each other in at least one translational and one rotational degree of freedom via an articulated coupling in such a way that an axis of the translational degree of freedom and a pivot point of the rotational degree of freedom lie in the area of the contact plates. A displacement-measuring device and a rotation-measuring device are arranged on the arms.

### REFERENCE TO RELATED APPLICATIONS

This application is a national stage filing under 35 USC 371 of International Application No. PCT/EP2007/001902, filed Mar. 6, 2007, which claims Convention priority from German Patent Application No. 20 2006 003 484.6, filed Mar. 6, 2006, the full disclosures of which are incorporated herein by reference.

### FIELD OF THE INVENTION

The invention relates to an instrument for measuring the stability of the cervical spine.

### BACKGROUND OF THE INVENTION

Instruments of this kind are used in operations for treating defects of intervertebral disks. One operating technique that has proven itself over the years involves the rigid connection of the vertebral bodies adjacent to the defective intervertebral disk. However, this reduces the mobility of the spinal column. This can cause considerable restriction, particularly in the neck area. Modern forms of treatment therefore aim to retain the functionality of the joint. To this end, cervical prostheses are known which are composed mainly of two cover plates, which are to be connected to the adjacent vertebrae, and of a joint component located between these. Depending on the structure of the joint, a distinction is made between two different types. One ensures complete natural freedom of movement, while the other limits the freedom of movement of the prosthesis joint. This type, also known as a constrained prosthesis, is used particularly in cases where there is poor stability of the spinal column. It is for the operating surgeon to decide which type of prosthesis to use. Since this depends on the individual pathology of the patient, the decision can generally be made only during the operation. This demands a great deal of experience.

An instrument for determining the range of mobility of a specific intervertebral disk has been described, in US 2004/0236342 A1, for the area of the lumbar spine, where the vertebrae are much larger than in the cervical spine. However, the appliance has a very complicated structure and takes up a lot of space. For example, twin scissor mechanisms are provided that are to be introduced into the intervertebral space. In practice, therefore, it is not really feasible for the appliance to be used in the area of the cervical spine, because of the small dimensions of the vertebrae there and because of the limited space.

For the cervical spine, appliances have been disclosed that measure the possible flexion/extension from outside the body and therefore allow conclusions to be drawn regarding the stability of the cervical spine. However, this entails a measurement across the entire cervical spine. It does not allow conclusions to be drawn concerning the degree of mobility in the area of a specific intervertebral disk between two adjacent vertebral bodies. This appliance permits only a global measurement, not a measurement focussed on the individual levels of the spinal column. Moreover, with this appliance, conclusions regarding the stability can only be made in the state prior to the operation. It is not possible to tell how great the stability will be after the removal of ligaments, located in the access route, and of the joint capsule.

### SUMMARY OF THE INVENTION

The object of the invention is to make available an instrument which is used for measuring the stability of the cervical spine and which avoids the abovementioned disadvantages and can be used during surgery.

The solution according to the invention lies in the features of the invention as broadly described herein. Advantageous developments are the subject matter of the preferred embodiments.

According to the invention, an instrument for measuring the stability of the cervical spine has two arms with in each case, at one end, a contact plate, and, at an opposite end, an actuating member, the arms being movable relative to each other in at least one translational and one rotational degree of freedom via an articulated coupling, in such a way that an axis of the translational degree of freedom and a pivot point of the rotational degree of freedom lie in the area of the contact plates.

The core of the invention is the provision of such a coupling that permits both a rotational and also a translational movement between the contact plates. By means of the actuating member, the arms can be displaced lengthwise relative to each other or can be moved at an angle to each other about a pivot point in the area of the contact plates. The contact plates each lie with their outwardly directed surface on the top face and bottom face, respectively, of the adjacent vertebral body lying above or below. With the two contact plates, the instrument can be pushed into the intervertebral space that has been freed of a defective intervertebral disk of the cervical spine and in which a joint prosthesis is intended to be implanted. It is thus possible for the operating surgeon to determine the flexibility and stability of the cervical spine in respect of a translational movement in the plane of the contact plates in which the arms are displaced relative to each other in the AP and/or lateral direction, and also the stability of the cervical spine in respect of a rotational movement, as occurs, for example, when nodding or extending the head (flexion or extension). From these two measurements, the operating surgeon is able to form a picture of the stability. The measurement is carried out precisely between the two vertebral bodies between which the joint prosthesis is also to be inserted. The measurement is thus performed exactly at the intended site of implantation. The measurement is also carried out after opening up the operating site and freeing the intervertebral space, which process involves removal of the joint capsule and, if appropriate, any ligaments obstructing the access route. The measurement can thus be carried out during the operation, specifically under the same conditions applying to the joint prosthesis that is to be inserted. A deterioration in the stability of the cervical spine, as may be caused by the removal of ligaments, for example, is in this way taken into consideration. With the instrument according to the invention, the operating surgeon is thus provided with valuable measurements of the stability of the cervical spine. Based on these measurements, he is able to decide, even during the operation, whether a joint prosthesis with complete or limited freedom of movement should be implanted. The instrument according to the invention thus combines the advantages of being able to be used during an operation and the advantages of a high degree of precision targeted specifically at the implantation site.

The contact plates preferably have a similar design to the cover plates of the prosthesis that is to be implanted. The contact plates are expediently oriented parallel to each other. An interface between the contact plates expediently lies in a mid-plane of the instrument, and the relative longitudinal mobility of the arms lies in a second plane (sagittal plane) which is perpendicular to the mid-plane and intersects it in a longitudinal axis of the instrument. This choice of the planes has the effect that the two contact plates can be displaced in translation relative to each other along the interface. The sagittal plane is also a tangential plane of the rotational degree of freedom. This permits measurement in the two stated degrees of freedom.

The articulated coupling can be designed for direct or indirect connection of the two arms. Indirect is here understood as meaning that the connection is made via adjacent vertebral bodies. For example, a rotary bearing of the articulated coupling can be formed via adjacent vertebrae, in which case the contact plates in the inserted state bear in a rotationally fixed manner on the vertebral bodies. The rotary bearing is expediently configured such that its axis of rotation lies transverse to the longitudinal axis of the instrument in the mid-plane, that is to say intersects the axis along which the translational movement takes place. This applies irrespectively of whether the rotary bearing is configured indirectly via vertebral bodies or directly as a structural element, for example a pivot pin. The articulated coupling also expediently comprises a longitudinal bearing with guide surfaces along the longitudinal axis. This provides a guide for a longitudinal displacement of the two arms relative to each other. It is particularly expedient to configure the longitudinal bearing and rotary bearing combined with each other.

With the instrument according to the invention, the surgeon will often already be able to decide on the prosthesis type on the basis of the impression gained using the instrument. However, it is also often desirable to have an objectively quantified measure available. For this purpose, the instrument according to the invention is expediently provided with a displacement-measuring device and a rotation-measuring device. It is expediently arranged on the arms. In the preferred embodiment, the displacement-measuring device is designed as a scale on one of the arms and as an index, preferably with a vernier, on the other of the arms. In this way, a quantitative measure of the displaceability in the longitudinal direction can easily be obtained. Moreover, the rotation-measuring device can be designed, on one of the arms, as a scale that is curved concavely to the contact plates and, on the other arm, as a second index. It has proven advantageous to arrange the second index on an arm that is guided in a slit in the scale. In this way, it is possible to prevent the arms from coming apart.

The contact plates are preferably provided with ribs on their surface that bears on the vertebral bodies. This protects them from inadvertent displacement relative to the vertebral bodies. It is thus possible to prevent measurement errors, which could occur in measuring the longitudinal displaceability, for example. In order to ensure greater safety against undesired movement of the contact plates relative to the vertebral bodies, in particular during the angle measurement, locking tabs are preferably provided, which can move from a rest position, in which they are recessed into the contact plate, to a locking position in which they protrude from it. In the locking position, the tabs engage in the surface of the vertebral body and in this way ensure a secure hold.

In a further development, the articulated coupling is expediently formed in such a way that the arms are movable relative to each other about a further rotational degree of freedom, and that an add-on angle measurement device is provided. The add-on angle measurement device can be combined with the already mentioned angle-measuring device. With the additional rotational degree of freedom, a possible tilting of the cervical spine in a second plane, for example with respect to a lateral inclination, can be determined. This permits an even more comprehensive assessment of the stability of the cervical spine. With this further development, the instrument according to the invention permits a measurement in three degrees of freedom.

To be able to perform the measurement in a defined manner either with respect to one or other rotational degree of freedom, a neutral position is expediently provided from which the arms are movable either along the first rotational degree of freedom or the second rotational degree of freedom. A combined movement is thus ruled out. In this way, measurements in the two rotational degrees of freedom can easily be carried out separately from each other, such that the results can be assigned unambiguously to the respective degree of freedom.

### BRIEF DESCRIPTION OF THE DRAWINGS

The invention is explained below with reference to the attached drawing which depicts an advantageous illustrative embodiment and in which:

FIG. 1 shows a plan view of a first illustrative embodiment of an instrument according to the invention;

FIG. 2 shows a side view of the instrument depicted in FIG. 1;

FIG. 3 shows a side view during a translational movement;

FIG. 4 shows a side view during an extension movement;

FIG. 5 shows a front view of the extension movement depicted in FIG. 4;

FIG. 6 shows a detail corresponding to FIG. 5, for a second illustrative embodiment of the invention; and

FIG. 7 shows two views of guide surfaces according to the first and second illustrative embodiment.

### DETAILED DESCRIPTION OF THE INVENTION

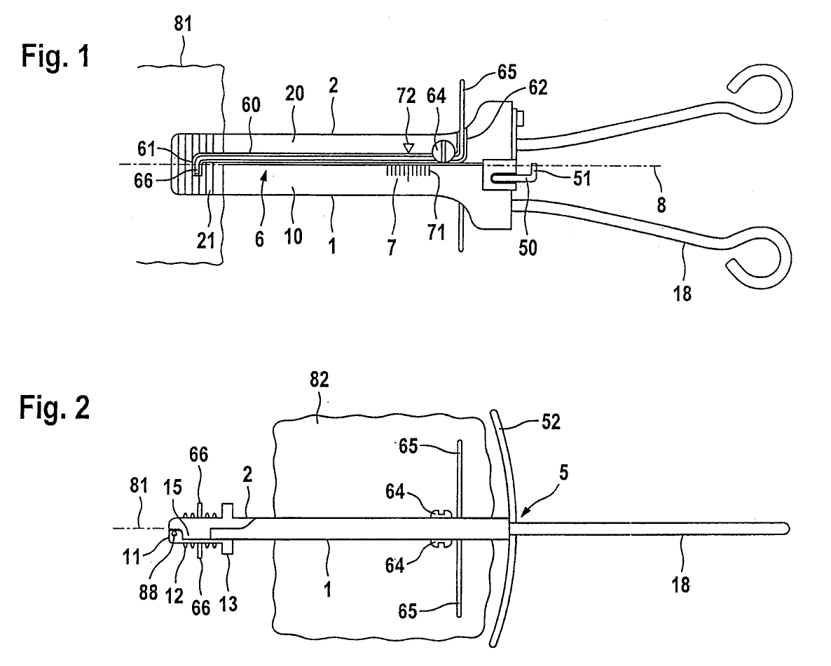

The first illustrative embodiment shown in FIG. 1 for an instrument according to the invention comprises, as its main components, two arms 1, 2. Both are largely identical in terms of their basic structure, but differ in terms of some elements of their angle-measuring device, which will be explained in detail below. The structure of the arms 1, 2 is explained below, taking the arm 1 as an example.

The arm 1 comprises a shaft 10 with a contact plate 11 arranged at its front end and a handgrip 18 arranged at its rear end. The shaft 10 has an approximately rectangular cross section with a top face, a bottom face and two side faces. A scale 71 of a length-measuring device 7 is arranged on the top face. The contact plate 11 ends flush with the underside of the arm 1. The contact plate 11 is only about half the thickness of the shaft 10, but is approximately twice as wide. On its outer face (which is flush with the bottom face of the shaft 10), it is designed for contact with an end face of an adjacent vertebral body 9. For this purpose, its surface has a plurality of ribs 12. In the area of transition between the contact plate 11 and the shaft 10, there is an abutment flange 13 that extends transverse to a longitudinal axis 8 of the instrument. It serves to limit the depth of insertion of the instrument with its contact plates 11, 21 into the space between two adjacent vertebrae 9. The handgrip 18 is made from a circular wire material with a hook at the end. It extends rearward substantially along the longitudinal axis 8, the outer end diverging outward relative to the longitudinal axis 8. This serves firstly for better adaptation to the anatomy of the surgeon's hand and thus for improved grip and maneuverability. Secondly, the resulting greater distance between the two handgrips 18 of the two arms 1, 2 allows a greater torque to be applied.

A locking device 6 is arranged on each of the arms 1, 2. FIG. 1 shows the locking device 6 of the arm 2. The following description relates to the latter; the same applies with respect to the other arm 1. It comprises a groove-like depression 60 formed in the underside of the shaft 20. This depression 60 extends substantially parallel to the longitudinal axis 8, with two 90° bends 61, 62 at the ends. The rear bend 62 opens in the side face of the shaft 20. A flexurally stiff wire 65 that is bent twice is introduced into the recess 60. It is held permanently in place by means of a screw 64, but is rotatable in the recess 60. At its two ends, the wire 65 has wing-like bends. The front bend is designed as a locking scoop 66. It lies in a rest position in the bend 61, such that it does not protrude from the contact plate 21. In its locking position, it projects perpendicularly from the surface of the contact plate 21. To actuate the wire 65, the rear protruding wing is designed as a handle. The surgeon can use it to actuate the wire in such a way that the locking tab 66 is in its rest position or in its locking position. A locking device 6 of a similar kind is arranged in the arm 1. The two locking devices 6 can be operated independently of each other via the respective handle.

An angle-measuring device 5 is arranged at the handle end of the arm 10. It mainly comprises a display unit 52 and an angle index 51. The display unit 52 is secured fixedly on the rear face of the arm 2 by means of a screw. It has a slot-like recess 56. A scale 54 is arranged laterally thereon and indicates an angular deviation from the longitudinal axis 8. The angular deviation is indicated by the angle index 51. The latter is arranged on a branch 50 on the rear face of the shaft 10 of the arm 1 in such a way that it extends through the slot-like recess 56 and has a display marker 53 (designed as nose) at its free end. The branch 50 pushed through the slot-like recess 56 additionally guides the arm 1 on the arm 2. This prevents the arms 1 and 2 from coming apart. With the arm 1 diverging, the relative angle setting between the arms 1 and 2 can easily be read off from the scale 54 of the angle-measuring device 5 using the marking 53 of the angle index 51.

The two contact plates 11, 21 are separated by a common plane interface 81. The interface lies in a plane with the longitudinal axis 8 and the handles 18 arranged centrally on the end faces of the arms 1, 2. Along this interface 81, the contact plates 11 of the two arms 1, 2 can be displaced relative to each other. For this purpose, lateral guide plates 15 are provided on the contact plates 11, 21. The side faces of the arms 1, 2 are correspondingly separated from each other by a second plane interface, a sagittal face 82. The side faces of the arms 1, 2 are designed in such a way that the two arms 1, 2 are movable relative to each other in this sagittal face 82. The sagittal face 82 is perpendicular to the interface 81, these intersecting in the longitudinal axis 8 (see FIGS. 7a, b). This orthogonal arrangement of the interface 81, on the one hand, and of the sagittal face 82, on the other hand, means that the movement of the two arms 1, 2 of the instrument according to the invention is permitted both in a translational degree of freedom and also in a rotational degree of freedom. This is explained in more detail below.

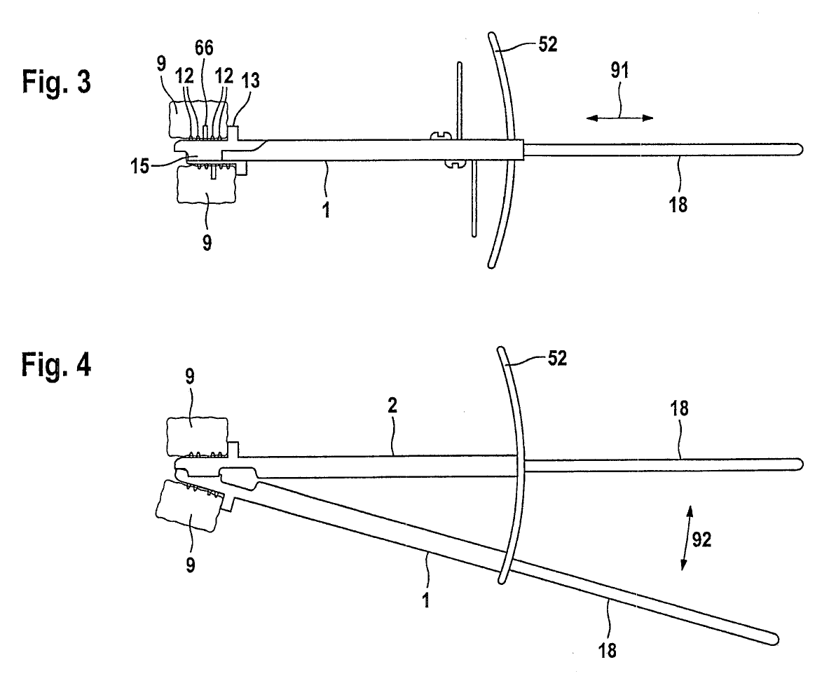

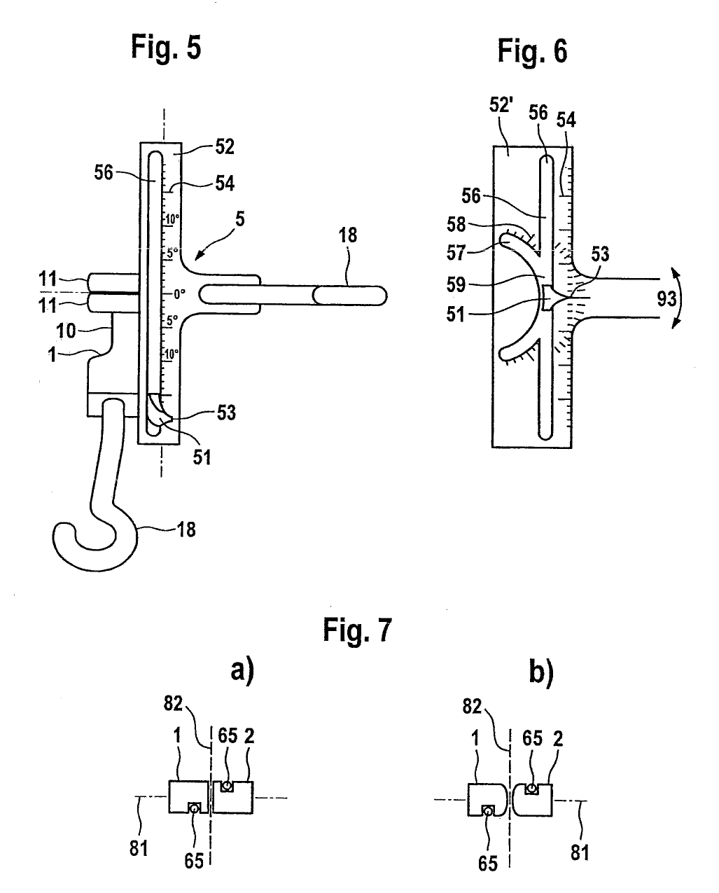

FIG. 3 shows how, starting from the normal position depicted in FIGS. 1 and 2, the two arms 1, 2 are moved in a translational degree of freedom (symbolized by a double arrow 91). It is assumed here that the instrument according to the invention is inserted into a space between two adjacent vertebral bodies 9. The contact plates 11, 21 thus bear with their respective outer surfaces on faces of the vertebral bodies 9. The ribs 12, and the locking tabs 66 brought into their locking position, secure the contact plates 11, 21 against slipping relative to the vertebral bodies 9. The locking is achieved by actuating the handle 65 of the locking devices 6 after insertion of the instrument, as a result of which the locking tab 66 pivots out from the contact plate 11, 21 and engages in the face of the respective vertebral body 9. The instrument is thus in its measurement position and ready for measurement.

The measurement of the translational displaceability is shown in FIG. 3. By means of the handgrips 18, the arms 1, 2 are moved relative to each other in the direction of the longitudinal axis 8. To be more precise, the arm 1 is drawn rearward (to the right in FIG. 3). In this way, the contact plates 11, 21 of the arms 1, 2 move along the interface 81. Since the contact plates 11, 21 are connected to the adjoining vertebral bodies 9 via the ribs 12 and the locking tabs 66 so as to be free from slipping, the vertebral bodies 9 are likewise displaced relative to each other in this direction. By means of the length-measuring device 7 arranged on the upper face and provided with the scale 71 and the index 72, it is possible to determine the relative translational movement, effected by the respective force, of the vertebral bodies 9 between which the contact plates 11, 21 are inserted. It will be appreciated that the length-measuring device 7 does not necessarily have to be configured mechanically as shown. It is equally possible for a measurement transducer to determine the relative longitudinal movement between the arms 1, 2 and to output this on a remote display. It is generally the case that the operating surgeon uses his or her feel to determine the force with which to effect the translational movement. However, if it is desirable to have a greater degree of precision or a high degree of reproducibility, for example for reports, provision can also be made for a force-measuring device to be arranged between the handgrip 18 and the arms 1, 2. In this way, the introduction of defined forces can be monitored.

A further step involves determining a rotational degree of freedom (symbolized by a double arrow 92). In FIG. 4, this is illustrated using the extension of the spinal column as an example. In contrast to the translational measurement depicted in FIG. 3, in the measurement of extension (or also accordingly in the measurement of flexion) the arms 1, 2 are not displaced relative to each other along the longitudinal axis 8; instead, one of the two arms (in FIG. 4 the arm 1) is deflected out of the rest plane about a defined angle. The force needed for this purpose is exerted by the operating surgeon once again via the handgrips 18. As has already been explained above, the surgeon's feel is generally sufficient to determine the degree of force, although a force-measuring device can also be used. By pressing the handgrip 18 of the arm 1 down, this arm is deflected downwards. The point of rotation lies in the area of the contact plates 11, 21, more precisely in the front area in the interface 81 on the longitudinal axis 8. The adjacent vertebral bodies 9 are inclined relative to one another, corresponding to a movement during extension (the same would apply upon flexion by moving the arm 1 in the opposite direction). The angular deflection arising from introduction of a defined force (or torque) can be determined via the angle-measuring device 5.

By determining the stability both by a translational movement 91 (see FIG. 3) and also by a rotational movement 92 (see FIG. 4), the operating surgeon can easily determine the stability of the spinal column, focussing on the level between the two adjacent vertebral bodies 9. This is done during the operation. Changes required intraoperatively to the spinal column, in particular sectioning of ligaments, are taken into account. In this way, the instrument according to the invention permits measurement that is both precise and also spatially focussed.

Upon divergence of the arms 1, 2 in the direction of the rotational degree of freedom, the side faces of the shafts 10, function as guide surfaces (see FIG. 7). These surfaces are usually configured as planes (see FIG. 7a). However, provision can also be made for them to be rounded (see FIG. 7b). The latter configuration affords the additional advantage that a second rotational degree of freedom is permitted.

A second illustrative embodiment of the instrument according to the invention has an additional rotational degree of freedom 93. This rotational degree of freedom 93 permits rotation about the longitudinal axis 8 (the actual rotation axis is in most cases slightly offset toward the respective handgrip 18). For this purpose, the angle-measuring device 5 is refined in such a way that it has a second angle-measuring device for the second rotational degree of freedom 93. A second slot-shaped recess 57 is provided. It is shaped like an arc of a circle and intersects the already mentioned slot-like recess 56 for the first rotational degree of freedom 92 in the area of a neutral position 59. A second scale 58 is arranged on the slot-shaped recess 57 shaped like an arc of a circle. With this scale, it is possible, by means of the angle index marker 51, to read off a tilt angle of the instrument according to the invention. The position of the index marker 51 shown in FIG. 6 is the neutral position 59. From the latter, the instrument can be moved along both the first and second rotational degrees of freedom 92, 93, that is to say along the slot-shaped recess 56 or 57. If the arm 1 is deflected in one of the two rotational degrees of freedom 92, 93, it remains bound to this until it is guided back into the neutral position 59.

FIG. 7b shows the configuration of the side surfaces of the arms 1, 2 for the second illustrative embodiment. The sagittal surface 82 is unchanged from the first illustrative embodiment. The side surfaces of the arm shafts 10, 20, however, are not plane, but convexly rounded. This permits a mutual tilting of the arms 1, 2. In this way, the stability of the spinal column with respect to lateral tilting movements can be determined. The surgeon thus acquires an additional parameter for determining the stability. The reliability of the determination is thus enhanced.

The invention can be summarized as follows. The invention relates to an instrument for measuring the stability of the cervical spine. It has two arms 1, 2 which, by means of an articulated coupling, are connected in such a way that they are movable relative to each other both in a translational degree of freedom and a rotational degree of freedom. Measuring devices 5, 7 are provided in order to determine the respective excursion in the translational movement or rotational movement. The instrument has, at its front end, contact plates 11, 21 designed to engage in an intervertebral space. By moving the arms 1, 2 of the instrument relative to each other, the vertebral bodies 9 are displaced or tilted relative to each other via the contact plates 11, 21. In this way, the operating surgeon is easily able to determine, in a reproducible manner, the stability of the cervical spine in the area of these adjacent vertebral bodies 9.

1-14. (canceled)

15. An instrument for measuring the stability of the cervical spine, comprising two arms each having, at one end, a contact plate configured for insertion into a space between two adjacent vertebral bodies, and, at an end opposite to the one end, an actuating member,
the arms being movable relative to each other in at least one translational and one rotational degree of freedom via an articulated coupling in such a way that an axis of the translational degree of freedom and a pivot point of the rotational degree of freedom lie in the area of the contact plates.

16. The instrument of claim 15, wherein a displacement-measuring device and a rotation-measuring device are arranged on the arms.

17. The instrument of claim 15 or 16, wherein the articulated coupling is formed indirectly via adjacent vertebral bodies, the contact plates bearing on the vertebral bodies in a rotationally fixed manner in an inserted state.

18. The instrument of claim 15 or 16, wherein an interface between the contact plates lies in a mid-plane of the instrument, and the rotational degree of freedom has a tangential plane which is perpendicular to the interface and intersects the interface in a longitudinal axis of the instrument.

19. The instrument of claim 15 or 16, wherein the articulated coupling comprises a rotary bearing whose axis of rotation is normal to a tangential plane.

20. The instrument of claim 15 or 16, wherein the articulated coupling comprises a longitudinal bearing having a guide surface lying in a second plane.

21. The instrument of claim 16, wherein the displacement-measuring device is in the form of a scale on one of the arms and in the form of an index on the other of the arms.

22. The instrument of claim 16, wherein the rotation-measuring device is in the form of a scale curved convexly with respect to the contact plate on one of the arms and in the form of an angle index on the other of the arms.

23. The instrument of claim 22, wherein the angle index is arranged on an arm which is guided in a slit of the rotation-measuring device.

24. The instrument of claim 15 or 16, wherein the contact plates have a plurality of ribs on their outer faces.

25. The instrument of claim 15 or 16, further comprising locking tabs provided on the contact plates that can be moved from a rest position, in which they are recessed into the contact plates, to a protruding locking position.

26. The instrument of claim 15 or 16, wherein the articulated coupling is configured in such a way that the arms are movable about a second rotational degree of freedom, and the instrument further comprises an add-on angle measuring device.

27. The instrument of claim 26, wherein the add-on angle measuring device is combined with the rotation-measuring device.

28. The instrument of claim 26, wherein the arms are configured to move from a neutral position either along the first rotational degree of freedom or along the second rotational degree of freedom.
